SEQUENCE LISTING (7 sequences)

SEQ ID NO: 1 - DNA, 2043 bp, Streptomyces hygroscopicus
gtgctcgggctcaacggcaacttctccgccgcggacaccgatgtggtgccgcagctcggagaggtgttctttcatgactcggcggcttccttgatccgcgacggcgaactcgtggccgccgtggaggaggagcggctcaaccggatcaagaagacaaccaaatttcccctcaacgcggtccgtgagtgcctggccctggccggtgcgcggcccgaggacgtcgacgcggtgggctactactttcccgagaaccacatcgacaccgtcctcaaccacctctacaccgaatatccgcgggcacccctgcgctactcccgggagctgatccggcagcggctgaaggagggcctgggctgggacctgccggacgagaagctggtgtacgtgccgcaccacgaggcgcacgcgtactcctcgtatctgcactccggtatggactccgcactggtcctggtgctggacggccgtggcgaactgcactccggcaccgtctaccgcgccgagggcacgcggctggagaagctggccgactacccggtgcccaagtcgctcggcgggctctacctgaacgccacctatctgctcggctacggcttcggcgacgagtacaaggtgatgggtctggccccctggggcaacccggagacctaccgcgacaccttcgccaagctctacaccctccaggacaacggcgagtacgagctgcacggcaacatcatggtgccgaacctggtcagcccgctgttctacgccgagggcttccggccgcgccgcaagggcgagccgttcacccaagcgcaccgcgacttcgccgccgcgctccaggagacggtcgagaagatcgtgctgcacatcctcgaatactgggcgaagaccagcggccactcccgcctgtgcttcggcggtggcgtcgcccacaactccagcctcaacgggctgatcctcaagtccggactcttcgacgaggtgttcgtgcaccccgcctcgcacgacgcgggcgcgggcgagggcgccgcctacgccgcggcggcgagccttggcacgctggagcgcccggggaagcggctgctcagcgcgagcctcggcccggcactgggcggccgggagcagatcagggcacggttggccgactgggcgccgctgatcgatgtggagttcccggacgacgccgtggagaccgcggccggactcctcgccgagggacaggtgctcggctgggcgtacggccgctccgagttcggcccccgcgccctgggccaccgcagcatcgtcgcggacgcacgccccgaggagaaccggacccgcatcaacgcgatggtgaagaagcgcgagggcttccggccgttcgccccggtggtcaccgccgaagccgcccgcgactacttcgacctctccggcgcggacggcaaccacgagttcatgtccttcgtggtgccggtgctgccggagcggcgtacggaactcggcgcggtcacccacgtggacggcaccgcccgggtacaggtcgtctccgccgagtccggcgagcggttccaccgcctggtgcggcggttcggcgaactgaccggcacccccgtgctcctcaacacctccttcaacaacaacgccgaacccatcgtgcagagcctcgacgacgtggtcaccagcttcctgaccaccgacctggacgttctggtggtggaggactgcctggtacgcggcaaagcctcgcccgacctgggcgttctggtgccgcggttccgcccggtgacccggctggtcgagcgccggacggccggtccggacgcctcggcgggagccaagacccacgagatccacctcgactacgacggcggcccgtccgcgaaggtgtcgcccgagctgtacgaactgctcggcgcggtcgacggcaccaccaccctcggggatctggccaagaccgtgggcgggctgtcggacgcactggccaccgaggtgttcgccctgtgggagcagcggttcctcaccctggccccggccggggacgtagggccgttggccgacgacggtacgcgggggcactga

SEQ ID NO: 2 - DNA, 31 bp, Artificial Sequence
  primer
gagatctcttgtgctcgggctcaacggcaac

SEQ ID NO: 3 - DNA, 25 bp, Artificial Sequence
  primer
aactccacatcgatcagcggcgccc

SEQ ID NO: 4 - DNA, 25 bp, Artificial Sequence
  primer
gactgggcgccgctgatcgatgtgg

SEQ ID NO: 5 - DNA, 25 bp, Artificial Sequence
  primer
atcgggtcagtgcccccgcgtaccg

SEQ ID NO: 6 - DNA, 23 bp, Artificial Sequence
  AHBA primer
ccsgccttcaccttcatctcctc

SEQ ID NO: 7 - DNA, 23 bp, Artificial Sequence
  AHBA primer
tcatcagcttgccgttctggaag

(19) United States
(12) Patent Application Publication
(10) Pub. No.: US 2008/0275039 A1
(43) Pub. Date: Nov. 6, 2008

(54) Geldanamycin Derivatives and the Method for Biosynthesis Thereof

(76) Inventors: Jung Joon Lee, Taejeon-si (KR); Young Soo Hong, Taejeon-si (KR); Dongho Lee, Taejeon-si (KR); Jeong-Hyung Lee, Taejeon-si (KR); Kwang-Soo Sung, Chungcheongnam-do (KR)

Correspondence Address: LUCAS & MERCANTI, LLP, 475 PARK AVENUE SOUTH, 15TH FLOOR, NEW YORK, NY, 10016, US

(21) Appl. No.: 11/573,503

(22) PCT Filed: Aug. 11, 2005
(86) PCT No.: PCT/KR2005/002601

§ 371 Date: Feb. 9, 2007

(30) Foreign Application Priority Data
Aug. 11, 2004 (KR) 10-2004-0063106

(51) Int. Cl. A61K 31/5377; C07D 225/06; C07H 21/04; C12N 1/20; C12P 17/10; C12N 15/64; A61P 31/04; A61P 37/06; A61P 35/00; A61P 31/12; A61P 31/10

(52) U.S. Cl. 514/234.5; 540/461; 536/23.1; 435/252.35; 435/121; 435/320.1

(57) ABSTRACT

The present invention relates to geldanamycin derivatives, benzoquinone ansamycin biosynthesized by gene manipulation of Streptomyces hygroscopicus subsp. duamyceticus and the method producing them, more particularly to a geldanamycin O-carbamoyl transferase gene(gel8)-inactive mutant, the method producing it and geldanamycin derivatives, 4,5-dihydro-7-O-descarbamoyl-7-hydroxy geldanamycin and 4,5-dihydro-7-O-descarbamoyl-7-hydroxy-17-O-demethyl geldanamycin. Since geldanamycin derivatives of the present invention suppress Hsp90 like geldanamycin, they can effectively be used for antibiotic, antifungal, antiviral, anti-inflammatory and antitumor agents and an immune suppressant.

### TECHNICAL FIELD

The present invention relates to geldanamycin derivatives and a method for biosynthesis thereof, more precisely, geldanamycin derivatives, benzoquinone ansamycin biosynthesized by geldanamycin O-carbamoyl transferase gene (gel8)-inactive mutant of Streptomyces hygroscopicus subsp. duamyceticus strain and a preparation method thereof.

### BACKGROUND ART

Geldanamycin, along with herbimycin, macbecin and is reblastatin, is a chemical compound having a polyketide backbone structure which is biosynthesized by using 3-amino-5-hydroxybenzoic acid (AHBA) as an initial precursor. The above compounds were proved to have functions of antibiotic, antifungal, antiviral and anticancer agents ((C. DeBoer et al. J. Antibiot. 1970, 23, 442-447. S. Omura, et al. J. Antibiot. 1979, 32, 255-261. M. Muroi et al. J. Antibiot. 1980, 33, 205-212, L. Neckers et al. Invest. New Drugs 1999, 17, 361-373).

Geldanamycin is a 19-membered macrocyclic lactam and is related to ansamycin antibiotics, such as rifamycins and ansamitocins. The biosynthesis of this class of compounds involves the assembly of 3-amino-5-hydroxybenzoic acid (AHBA), as a starter unit, followed by the sequential addition of extender units such as acetate, propionate and glycolate, to form a polyketide backbone, which then undergoes further downstream processing.

It was confirmed in 1994 by Neckers et al that the geldanamycin is conjugated to ATP binding site of heat shock protein 90 (Hsp90) having the activity of protein chaperone (L. Whitesell, et al, Proc Natl Acad Sci U.S.A. 1994, 91: 8324-8328). By the above founding, it was also confirmed that the anticancer effect of geldanamycin is generated not by inhibiting the enzymatic activity of tyrosin kinase having the function of oncogenic protein, but by inhibiting the functions of Hsp90 which is a crucial factor involved in structural stability of Hsp90 client proteins including tyrosine kinase.

Based on the physiological importance of Hsp90, geldanamycin and its derivatives and Hsp90 inhibitors such as radicicol and novobiocin have been used for the development of an anticancer agent (Peter W. Piper, Current opinion in Investigational Drugs, 2001, Vol 2, 1606-1610).

A gene cluster containing type-I polyketide synthase (PKS) involved in biosynthesis of geldanamycin was already cloned from other kinds of Streptomyces, and nucleotide sequence thereof was also identified. For example, U.S. patent application Ser. No. 10/461,194 describes that a recombinant polyketide synthase and polyketide modified protein can be produced by manipulating a gene involved in biosynthesis of geldanamycin in Streptomyces hygroscopicus var. geldanus NRRL3602 strain. And, benzoquinon ansamycin-like compounds which are useful for the treatment of cancer or other diseases caused by over-proliferation of unwanted cells and a preparation method thereof are described in U.S. patent application Ser. No. 10/212/962.

Korean Patent Application No. 2003-7008551 describes a novel geldanamycin derivatives and preparation method thereof under the title of “Geldanamycin derivatives and a treatment method for cancer using the same”, and Korean Patent Application No. 2004-7004202 describes chemical synthesis of 17-allyl amino geldanamycin (17-AAG) and other ansamycins under the title of “Preparation method of 17-allyl amino (17-AGG) and other ansamycins”.

Based on predictions from sequence homology and the results of feeding experiment with 14C-labeled precursor, it was proposed that the successful production of geldanamycin requires the modification of several steps, which including the O-carbamoylation, hydroxylation, O-methylation and oxidation of the initial polyketide synthase product (A. Rascher er al, FEMS Microbiology Letters 2003, 218: 223-230). However, beyond determining sequences and deducing putative functions from sequence homologies, little had been learned about the post-PKS modification genes and the tailoring processes leading from initial polyketide to geldanamycin.

Thus, taking notice of that geldanamycin or its' derivatives are able to be used as a therapeutic agent for the treatment of diseases including cancer, the present inventors studied on a method for mass-production of geldanamycin derivatives by manipulating the functions of a gene involved in the geldanamycin biosynthesis. As a result, the present inventors completed this invention by confirming that the method of the invention is very effective for the mass-production of geldanamycin or its derivatives.

### DISCLOSURE

### Technical Problem

It is an object of the present invention to provide a geldanamycin O-carbamoyl transferase gene(gel8)-inactive mutant, a preparation method of the same, novel geldanamycin derivatives derived from the mutant and a preparation method thereof.

### Technical Solution

To achieve the above object, the present invention provides a geldanamycin O-carbamoyl transferase gene(gel8)-inactive mutant derived from Streptomyces hygroscopicus subsp. duamyceticus and geldanamycin derivatives biosynthesized by the mutant.

The present invention also provides a preparation method of geldanamycin derivatives manipulating geldanamycin O-carbamoyl transferase gene of Streptomyces hygroscopicus subsp. duamyceticus.

Hereinafter, the present invention is described in detail.

The present inventors found post-PKS modified genes through nucleotide sequencing of gene cluster involved in geldanamycin biosynthesis, and among these genes, gel8 was proved to be a gene encoding carbamoyl transferase-like protein, which is responsible for O-carbamoylation in biosynthesis of novobiocin, ansamitosin and cephamycin.

Gel8-inactive mutant of the present invention is prepared by the following steps; gel8 gene which is inactivated by the insertion of a gene disruption construct is introduced into Streptomyces hygroscopicus duamyceticus, and the wild type is transformed into inactive mutant by homologous recombination.

Gel8 gene of the present invention has nucleotide sequence represented by SEQ. ID. No 1 and gel8-inactive construct is prepared by inserting other DNA like antibiotics resistant gene in the middle of gels gene.

DNA sequence possibly inserted into gel8 gene is an antibiotics (kanamycin, thiostrepton, etc) resistant gene.

The present invention further provides a recombinant vector pKC-CT containing the above gel8-inactive construct.

The present invention also provides a Streptomyces hygriscopicus AC1 (Accession No: KCTC 10675BP), the mutant of Streptomyces hygroscopicus subsp. duamyceticus, transfected with the above pKC-CT recombinant vector.

In the present invention, the mutant was cultured to obtain geldanamycin derivatives 4,5-dihydro-7-O-descarbamoyl-7-hydroxygeldanamycin (compound 3) and 4,5-dihydro-7-O-descarbamoyl-7-hydroxy-17-O-demethylgeldanamycin (compound 4) having the following formula.

The mutant of the present invention was grown up normally in YEME medium containing kanamycin and showed similar characteristics to those of the wild type. However, the mutant of the invention did not produce geldanamycin (compound 1) and 17-O-demethylgeldanamycin (compound 2), which are major metabolites of the wild type.

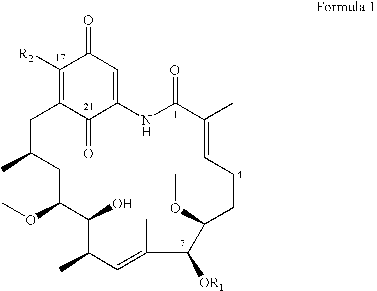

Wherein, R₁ and R₂ are defined as shown in Table 1.

TABLE 1

| | R₁ | R₂ | C₄—C₅ |
|---|---|---|---|
| Compound 1 | CONH₂ | OCH₃ | Double bond |
| Compound 2 | CONH₂ | OH | Double bond |
| Compound 3 | H | OCH₃ | Single bond |
| Compound 4 | H | OH | Single bond |
| Compound 5 | CONH₂ | OCH₃ | Single bond |

Instead, two major metabolites, 3 and 4 (m/z 519 and 505, respectively) were detected and isolated from the gene-inactivated mutant. Compounds 3 and 4 displayed ESIMS patterns resembling those of compounds 1 and 2. An analysis of the 1D and 2D NMR spectra of 3 suggested that it is a derivative of 1. From the ¹H and ¹³C NMR spectra of 3, the upfield shift of C-7 signals at δ_{H} 3.86 (1H, d, J=6.0 Hz) and δ_{C} 78.23, indicated that 3 has a free hydroxy group at C-7 rather than a carbamoyl group, as expected. Furthermore, two olefinic methine signals (C-4 and C-5) of 1 were not detected, suggesting that its cis double bond had been hydrogenated. This was consistent with the molecular formula C₂₈H₄₁O₈N obtained by positive HRFABMS. A combination of COSY, HMQC, and HMBC NMR data was used to assign the ¹H and ¹³C NMR data unambiguously. Therefore, the structure of this new metabolite was elucidated as 4,5-dihydro-7-O-descarbamoyl-7-hydroxygeldanamycin (3). The ¹H and ¹³C NMR spectra of 4 were almost superimposable with those of 3, except for the absence of one phenolic methoxy signal in the later compound, and were consistent with the molecular formula C₂₇H₃₉O₈N obtained by positive HRFABMS. Accordingly, the structure of this new metabolite was determined as 4,5-dihydro-7-O-descarbamoyl-7-hydroxy-17-O-demethylgeldanamycin (4).

It was confirmed that gel8 encodes carbamoyl transferase by the accumulation of descarbamoyled compounds in gel₈-inactive mutant of the present invention.

As explained hereinbefore, the present invention provides geldanamycin derivatives synthesized by gene manipulation in Streptomyces hygroscopicus duamyceticus. In accordance with the results of conventional analysis, these derivatives have not only anticancer activity but also other related physiological activities.

### DESCRIPTION OF DRAWINGS

FIG. 1 is a restriction enzyme map of 55-kb fraction of geldanamycin biosynthesis gene cluster obtained from Streptomyces hygroscopicus subsp. duamyceticus genomic cosmid DNA,

FIG. 2 shows the comparison of nucleotide sequences among three active sites of ER domains of geldanamycin PKS (polyketide synthase), animal fatty acid synthase and erythromycin PKS,

FIG. 3 shows the outlined processes of inactivation of geldanamycin O-carbamoyl transferase gene (gel₈) of geldanamycin biosynthesis gene cluster,

FIG. 4 is a graph showing the results of HPLC analysis with culture medium extract (No. 2) obtained from biotransformation of 4,5-dihydro-7-descarbamoyl-7-hydroxygeldanamycin with geldanamycin PKS gene-inactive mutant, culture medium extract (No. 1) obtained from geldanamycin PKS gene-inactive mutant and culture medium extract (No. 3) obtained from Streptomyces hygroscopicus subsp. duamyceticus JCM4427,

Arrow; Geldanamycin,

Asterisk; 4,5-dihydrogeldanamycin

FIG. 5 shows the outlined processes of geldanamycin biosynthesis,

FIG. 6 shows vector maps of pCR2.1-TOPO and pKC1139.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Cloning of Geldanamycin Biosynthesis Gene

Streptomyces hygroscopicus DNA library was constructed by using cosmid pOJ446 vector.

First, Streptomyces hygroscopicus (JCM4427) chromosomal DNA was obtained, which was partially digested with Sau3AI and dephosphorylated, followed by ligation into cosmid vector pOJ446 which was also dephosphorylated and digested with HpaI and BamHI (Bierm M et al., Gene 116: 43-49, 1992). Packaging of ligated products was performed with Gigapack III gold (Stratagene), which was transfected to E. coli XL-1 Blue MRF′ (Stratagene). Southern hybridization was performed (Sambrook, J., et al. Molecular Cloning: A Laboratory Manual, 2nd ed. Cold Spring Harbor Laboratory, Cold Spring Harbor, N.Y., 1989) with the prepared cosmid library by using fragments of AHBA synthase gene and polyketide synthase (PKS) gene.

More precisely for the hybridization, a cosmid enabling simultaneous hybridization of AHBA synthase gene and polyketide synthase gene was searched in consideration of the general structure of ansamycin antibiotics biosynthesis genes in which AHBA synthase gene and polyketide synthase gene existed as cluster. PCR product was performed with AHBA 1 primer (SEQ. ID. No 6) and AHBA 2 primer (SEQ. ID. No 7) and DNA fragment (Donadio S. et al. Science 252: 675-679, 1991) containing polyketide erythromycin biosynthesis gene were used as a probe. PCR was performed by using DNA polymerase (ExTaq polymerase, Takara) with the above mentioned primer sets and the template as follows; predenaturation at 95° C. for 5 minutes, denaturation at 95° C. for 1 minute, annealing at 60° C. for 1 minute, polymerization at 72° C. for 1 minute, 30 cycles from denaturation to polymerization, and final extension at 72° C. for 10 minutes. DMSO solution was added by 5% to enhance the reaction accuracy.

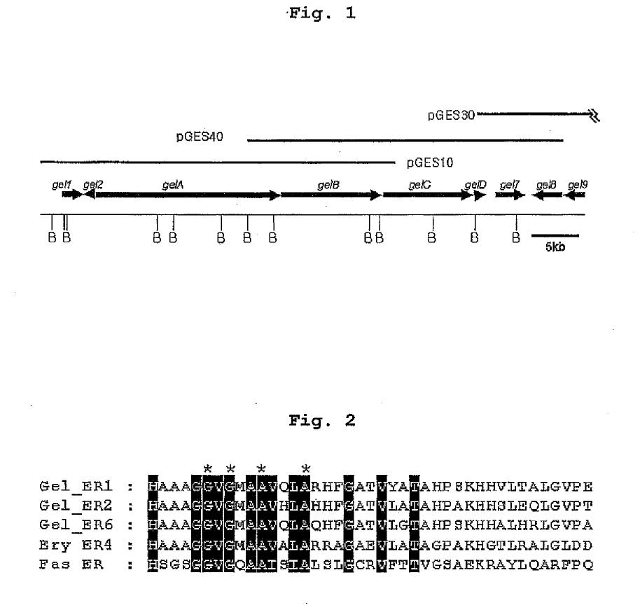

Cosmid clones identified in this manner were divided into two groups. Group 1 hybridized with both probes; group 2 hybridized with only the KS gene fragment (Hong Y S et al., 13^{th}.Int. Symp. on the Biology of Actinomycetes, Melborne, Australia, p33, 2003). Group 2 was further analyzed by comparative restriction enzyme mapping, as shown in FIG. 1, and was found to be identical to a previously reported geldanamycin biosynthetic gene cluster (Rasher A. et al., FEMS Microbiol. Lett. 81: 261-264, 1991). A total 55 kb fragment of cosmid 10 (pGES10) and 40 (pGES40) contained nine ORFs, with the following predicted functions; three PKS genes (gelA-C), an amide synthase gene (gelD), putative hydroxylase genes (gel1 & 7), and carbamoyl transferase (gel8) (FIG. 1.)

The deduced amino acid sequence of geldanamycin PKS displays significant homology to rifamycin and ansamitosin PKS. The enoylreductase (ER) domains were found in modules 1, 2, and 6. Module 6 of geldanamycin PKS contains a functional ER domain to reduce the double bond during polyketide assembly, as determined by sequence comparisons of the ER domain in module 6 in gelC with other functional ER domains. Putative NADPH binding sites, GxGxxAxxxA, of ER domains in animal fatty acid synthase and erythromycin PKS are well conserved in the corresponding ER domains of modules 1, 2, and 6 of geldanamycin PKS (FIG. 2).

Polyketide intermediates could be biosynthesized to have a structure lacking of double bond between C4 and C5 by geldanamycin PKS module 6, and then an enzyme inducing double bond between C4 and C5 was inserted, leading to the synthesis of geldanamycin.

### EXAMPLE 2

### Construction of gel8 Inactive Mutant

The present inventors constructed gel8 inactive mutant to investigate whether or not gel8 gene was involved in geldanamycin biosynthesis.

First, PCR was performed to amplify gel8 gene by using chromosomal DNA of Streptomyces hygroscopicus strain as a template with two pairs of primers (FIG. 2).

First pair, BglII restriction enzyme site was introduced,

| | |
|---|---|
| 5′-GAG [symbol] CTTGTGCTCGGGCTCAACGGCAAC-3′ | (Forward |
| | primer) |
| 5′-AACTCCACATCGATCAGCGGCGCCC-3′ | (Backward |
| | primer); |
| Second pair, | |
| 5′-GACTGGGCGCCG CTGATCGATGTGG-3′ | (Forward |
| | primer), |
| 5′-ATCGGGTCAGTGCCCCCGCGTACCG-3′ | (Backward |
| | primer). |

PCR was performed by using DNA polymerase (ExTaq polymerase, Takara) with the above mentioned primer sets and the template as follows; predenaturation at 95° C. for minutes, denaturation at 95° C. for 1 minute, annealing at 60° C. for 1 minute, polymerization at 72° C. for 1 minute, 30 cycles from denaturation to polymerization, and final extension at 72° C. for 10 minutes. DMSO solution was added by 5% to enhance the reaction accuracy.

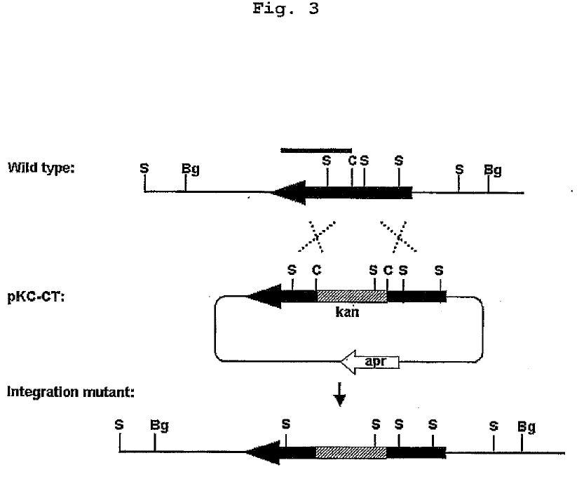

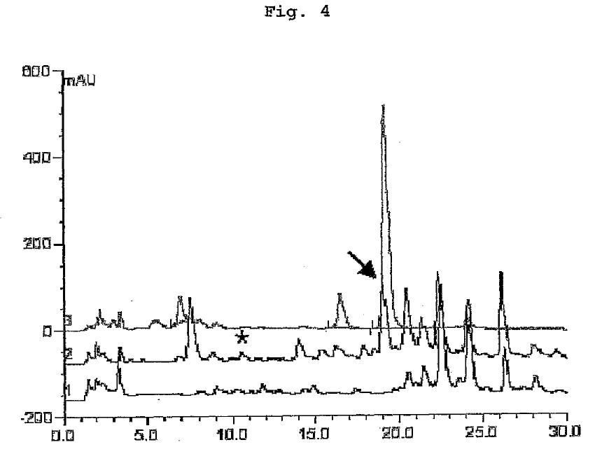

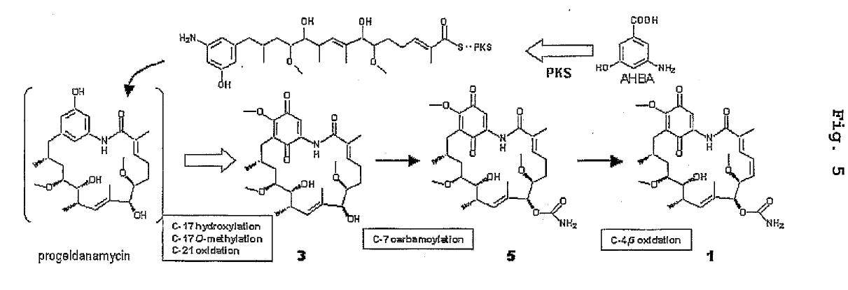

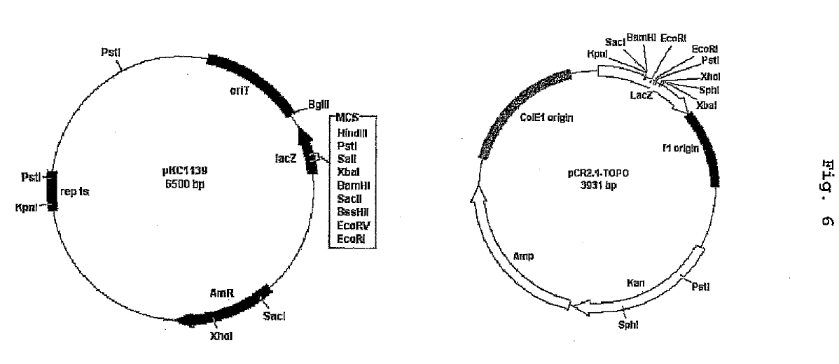

The PCR product was cloned into TA cloning vector pCR2.1-TOPO (Invitrogen™ life technologies), resulting in pCR-CTN and pCR-CTR. pFD-neoS 1.1-kb DNA fragment (Denis F and Brazezinski R. FEMS Microbiol. Lett. 81: 261-264, 1991) containing kanamycin resistant gene aphII was used to prepare a selection marker and a gene-destroy construct. For the gene-inactivation experiment, 1.1-kb BglII/KpnI fragment of pCR-CTN, 0.9-kb HindIII/XbaI fragment of pCR-CTR and 1.1-kb KpnI/HindIII fragment of pFD-neoS were ligated to pKC1139 (Bierman, M.; Logan, R.; O'Brien, K.; Seno, E. T.; Rao, R. N.; Schoner, B. E. Gene 116: 43-49, 1992) having apramycin resistance which was pre-digested with BamHI and XbaI, resulting in the construction of pCR-CT. This plasmid, pKC-CT, was then transformed into ET12567(pUZ8002) (Allen, I. W., and D. A. Ritchie., Mol Gen Genet. 243:593-599, 1994). Intergeneric conjugation between E. coli and Streptomyces was performed as described previously with minor modification (Flett, F., FEMS Microbiol. Lett. 155, 223-229, 1997). The transformant was resistance to both apramycin and kanamycin. The transformant was grown in fresh YEME/kanamycin liquid medium at 37° C. for 4 days in order to force chromosomal integration of pKC-CT. The kanamycin-resistant recombinants resulting from homologous recombination between the delivered vector DNA and wild-type S. hygroscopicus JCM4427 were selected from replica plates containing apramycin or kanamycin, and were resistant to kanamycin but sensitive to apramycin. Recombinants carrying disrupted gel8 were confirmed by PCR of their total genomic DNA. From the PCR was confirmed that carbamoyl transferase site of the total genomic DNA of the mutant was approximately 1 kb increased by the insertion of kanamycin. The produced strain was named Streptomyces hygroscopicus AC 1 and then deposited at KCTC on Aug. 4, 2004 (Accession No: KCTC 10675BP). The selected recombinant mutant had resistance against kanamycin but had sensitivity to apramycin (FIG. 3).

### EXAMPLE 3

### Products of the Wild Type and gel8 Inactive Mutant

### <3-1> Culture and Production Yield

S. hygroscopicus subsp. duamyceticus JCM4427 and gel8 inactive mutant (Streptomyces hygroscopicus AC) produced in the above Example 1 were cultured in 3 L of YEME medium for 5 days at 28° C. and the resultant products were accumulated. Upon completion of the culture, each medium was extracted with EtOAc twice and the extracts were filtered to eliminate insoluble substances. After concentration, fractionation with EtOAc and H₂O Was performed to give 2.1 g of extract from the wild type strain and 2.8 g of extract from the mutant produced in Example 1.

### <3-2> Separation of the Accumulated Compound

To identify the compound accumulated in Example 2, fractionation was performed by silica gel chromatography using CHCl₃-MeOH as moving phase. The obtained fractions were analyzed by TLC and ESIMS. Melting point was measured with Electrothermal 9100 without calibration. Specific rotation ([α]_{D}²⁵) and UV were measured respectively with JASCO DIP-370 polarimeter and Shimadzu UV-1601 spectrophotometer. Every NMR tests were performed with Bruker DMX 600 NMR spectrophotometer. ESIMS and HRFABMS were obtained respectively from Finningan LCQ Advantage Max mass spectrophotometer and JEOL JMS-HX110A/HX110A Tandem mass spectrophotometer. HPLC was performed by using Waters Delta Prep 3000 system.

From the HPLC analysis with each extract, compounds 1 and 2 (m/z 560 and 546) were detected in the wild type culture extract and compounds 3 and 4 (m/z 519 and 505) were detected in the mutant (Example 1) culture extract.

The fractions containing the extracted compounds 1 and 2, and compounds 3 and 4 were passed through Sephadex LH-20 column, followed by purification by HPLC [YMC J'sphere ODS-H80, 150 20 mm i.d., MeOH—H₂O (0.05% acetic acid) gradient, 10 mL/min]. The compounds 1 (t_{R} 19.2, 420 mg, 20% w/w) and 2 (t_{R} 16.6, 48 mg, 2.3% w/w) were obtained from the wild type culture extract and likewise, the compounds 3 (t_{R} 20.8, 480 mg, 17.1% w/w) and 4 (t_{R} 18.4, 30 mg, 1.1% w/w) were obtained from the mutant (prepared in Example 1) culture extract after purification by HPLC [YMC J'sphere ODS-H80, 150 4.6 mm i.d., MeOH—H₂O (0.05% acetic acid) 50:50 to 100:0 over 25 min, 1 mL/min]

### <3-3> Identification of the Accumulated Compounds (1,2,3 and 4)

The accumulated compounds were separated and analyzed, and the results were as follows.

Compound 1; yellow powder; mp 250-254 [symbol] [α]_{D}²⁵+60.4° (c 0.12, CHCl₃), UV (MeOH); λmax (log ε) 305 (4.10) nm ¹H and ¹³C NMR data, see Table 1; ESIMS m/z {561 [M+H]⁺, 559 [M−H]⁻}

Compound 2; yellow powder; mp 353-357 [symbol] [α]_{D}²⁵+50.0° (c 0.16, CHCl₃), UV(MeOH): λmax (log ε) 315 (4.20) am ¹H and ¹³C NMR data, see Table 1; ESIMS m/z {547 [M+H]⁺, 545 [M−H]⁻}

Compound 3; yellow powder; mp. 80-83 [symbol] [α]_{D}²⁵−6.7° (c 0.15, CHCl₃), UV(MeOH): λmax (log ε) 304 (4.24) nm ¹H and ¹³C NMR data, see Table 1; ESIMS m/z {520 [M+H]⁺, 518 [M−H]⁻} HRFABMS m/z 542.2732, C₂₈H₄₁O₈NNa calculated value, 542.2730.

Compound 4; yellow powder; rap 185-189 [symbol] [α]_{D}²⁵+30.3° (c 0.08, CHCl₃) UV(MeOH): λmax (loge) 310 (4.26) nm ¹H and ¹³C NMR data, see Table 11; ESIMS m/z {506 [M+H]⁺, 504 [M−H]⁻} HRFABMS m/z 528.2574, C₂₇H₃₉O₈NNa calculated value, 528.2573.

¹H and ¹³C NMR data of the compounds 1-4 are shown in Table 2.

From the results, it was confirmed that the accumulated compounds were respectively geldanamycin (compound 1), 17-O-dimethylgeldanamycin (compound 2), 4,5-dihydro-7-O-descarbamoyl-7-hydroxygeldanamycin (compound 3) and 4,5-dihydro-7-O-descarbamoyl-7-hydroxy-17-O-demethylgeldanamycin (compound 4).

### EXAMPLE 4

### Biotransformation of the Compound 3

To confirm whether or not the compound 3 obtained from gel8 inactive mutant was a convertible derivative, the present inventors constructed geldanamycin PKS inactive mutant which was inactivated by the insertion of loading domain (Hong Y S et al., 13^{th}.Int. Symp. on the Biology of Actinomycetes, Melborne, Australia, p33, 2003). The loading domain mutant did not produce geldanamycin but had complete post-PKS modification genes.

Biotransformation with the compound 3 was performed as follows. The loading domain mutant spores were inoculated to 250 ml baffled Erlenmeyer flask containing 30 ml of YEME medium, which was cultured at 28° C. for 3 days with 200 rpm. And then 3 mg of the compound 3 dissolved in 100 ul of EtOAc was added. The culture mixture was further cultured for 3 more days to induce the conversion of the compound 3 in a mutant (28 200-rpm). Culture medium was extracted twice with 30 ml of EtOAc. Among extracts, organic phase was vacuum-distillated. Remnants were dissolved in 100 ul of EtOAc to obtain products originated from the compound 3, which were identified by ESIMS (ESARR Implementation Monitoring and Support).

As a result, the geldanamycin (1) produced had the same ESIMS/MS profiles, including retention time, UV, molecular ion, and fragmentation pattern {559 [M−H]−→516 [M−CONH₂]−}, as those of authentic geldanamycin (Sigma, St. Louis, Mo.). In addition, the ESIMS/MS profiles of 4,5-dihydrogeldanamycin (5) were comparable to those of geldanamycin (1) including the UV and the fragmentation pattern of the molecular ion {561 [M−H]−→518 [M−CONH₂]−}.

### INDUSTRIAL APPLICABILITY

As explained hereinbefore, the present invention provides novel geldanamycin derivatives biosynthesized from a mutant generated by manipulating geldanamycin O-carbamoyl transferase gene involved in biosynthesis of geldanamycin of Streptomyces hygroscopicus subsp. duamyceticus and a preparation method thereof. And those geldanamycin derivatives have Hsp90 inhibitory activity which is similar to that of geldanamycin, so that they can be effectively used as an antibiotic, an antifungal agent, an antiviral agent, an immunosuppressant, an anti-inflammatory agent, and an anticancer agent.

### Sequence List Text

Nucleotide sequence represented by SEQ. ID. No 1 is the sequence of gel8 gene,

Nucleotide sequences represented by SEQ. ID. No 2 and No 3 are primer sequences used for the construction of pCR-CTN vector by amplifying gel8 gene using chromosomal DNA of a Streptomyces hygroscopicus strain as a template,

Nucleotide sequences represented by SEQ. ID. No 4 and no 5 are primer sequences used for the construction of pCR-CTR vector by amplifying gel8 gene using chromosomal DNA of a Streptomyces hygroscopicus strain as a template,

Nucleotide sequences represented by SEQ. ID. No 6 and No 7 are sequences of AHBA 1 primer and AHBA 2 primer used for the construction of probe for searching a hybridization cosmid.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A geldanamycin derivative represented by the following formula. 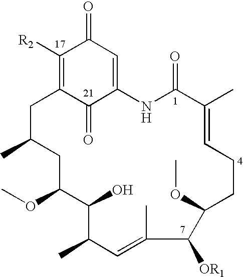
Wherein, R₁ is H, and R₂ is OH or OCH₃.

2. A gel8 gene having a nucleotide sequence represented by SEQ. ID. No 1.

3. A gel8 inactive construct prepared by inserting other DNA sequences into gel8 gene.

4. The gel8 inactive construct as set forth in claim 3, wherein the other DNA sequence is kanamycin resistant gene or thiostrepton resistant gene, which characteristically gives resistance against Gram-positive selection antibiotics to the construct.

5. A recombinant vector pKC-CT containing the gel8 inactive construct of claim 3.

6. A Streptomyces hygriscopicus AC1, which is a recombinant mutant of Streptomyces hygroscopicus subsp. duamyceticus, transfected with the pKC-CT recombinant vector of claim 5 (Accession No: KCTC 10675BP).

7. A method for the biosynthesis of 4,5-dihydro-7-O-descarbamoyl-7-hydroxygeldanamycin, a geldanamycin derivative of claim 1, by culturing the recombinant mutant of claim 6.

8. A method for the biosynthesis of 4,5-dihydro-7-O-descarbamoyl-7-hydroxy-17-O-demethylgeldanamycin, another geldanamycin derivative of claim 1, by culturing the recombinant mutant of claim 6.

9. An antibiotic containing the geldanamycin derivative of claim 1 as an effective ingredient.

10. An antifungal agent containing the geldanamycin derivative of claim 1 as an effective ingredient.

11. An antiviral agent containing the geldanamycin derivative of claim 1 as an effective ingredient.

12. An immunosuppressant containing the geldanamycin derivative of claim 1 as an effective ingredient.

13. An anti-inflammatory agent containing the geldanamycin derivative of claim 1 as an effective ingredient.

14. An anticancer agent containing the geldanamycin derivative of claim 1 as an effective ingredient.
